(19) United States
(12) Patent Application Publication
(10) Pub. No.: US 2022/0142981 A1
(43) Pub. Date: May 12, 2022

(54) APREMILAST LIPOPHILIC TOPICAL PHARMACEUTICAL COMPOSITIONS

(71) Applicant: APRAMITHA INNOVATIONS PRIVATE LIMITED, Secunderabad, Ranga, Reddy Telangana (IN)

(72) Inventors: Venkata Nookaraju SREEDHARALA, Hyderabad (IN); Ramdas MANAKKOTE, Hyderabad (IN); Srikanth KALAKOTI, Hyderabad (IN)

(73) Assignee: APRAMITHA INNOVATIONS PRIVATE LIMITED, Secunderabad, Ranga, Reddy, Telangana (IN)

(21) Appl. No.: 17/431,400

(22) PCT Filed: Feb. 17, 2020
(86) PCT No.: PCT/IN2020/050149

§ 371 Date: Aug. 16, 2021

(30) Foreign Application Priority Data
Feb. 19, 2019 (IN) 201941006472

(51) Int. Cl. A61K 31/4025; A61K 9/00; A61K 9/06; A61K 47/14

(52) U.S. Cl. CPC A61K 31/4025; A61K 47/14; A61K 9/06; A61K 9/0014

(57) ABSTRACT

The present invention provides a topical composition of Apremilast with lipohilic agents. The plurality of excipients each present in amounts, and in combination, to effectively control the release of Apremilast from the composition for better penetration in to the skin. The present invention also provides a process for preparation of the apremilast topical composition with enhanced skin penetration and efficacy. The present invention also relates to a method of treating psoriasis and other related skin condition using a lipophilic topical composition of Apremilast, wherein the permeation of Apremilast through the skin is significantly enhanced with the use of lipophilic components in the composition.

### FIELD OF INVENTION

The present invention relates to a lipophilic topical composition of Apremilast. The said composition has plurality of excipients in particular quantity and ratios, and in best suitable combination to effectively control the release of Apremilast from the composition and provide enhanced penetration in to the skin. The invention also provides composition for effective treatment of psoriatic symptoms and other related skin disorders.

### BACKGROUND

Apremilast is a selective inhibitor of the enzyme phosphodiesterase 4 (PDE4) and inhibits spontaneous production of TNF-alpha from human rheumatoid synovial cells. Apremilast is chemically known as N-[2-[(lS)-l-(3-ethoxy-4-methoxyphenyl)-2-(methylsulfonyl)ethyl]-l,3-dioxo-2,3-dihydro-lH-isoindol-4-yl]acetamide which can be characterized by the following chemical formula:

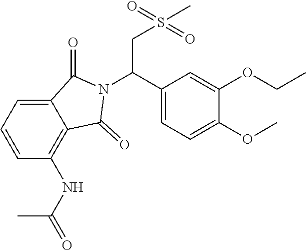 [SMILES: CCOc1cc(C(CS(C)(=O)=O)N2C(=O)c3cccc(NC(C)=O)c3C2=O)ccc1OC]

Apremilast is approved in United States of America and Europe, marketed by Celgene Corp as OTEZLA®. OTEZLA® tablets are supplied in 10, 20, and 30 mg strengths for oral Administration.

Apremilast being a selective phosphodiesterase 4 (PDE4) inhibitor acts by increasing the intracellular cyclic adenosine monophosphate (cAMP) which decreases the expression of inflammatory cytokines such as tumour necrosis factor and interleukin-23 (IL-23). It is indicated for the treatment of active psoriatic arthritis and plaque psoriasis. Commercially available forms of Apremilast are available as tablets of 10, 20 and 30 mg for oral administration.

U.S. Pat. No. 6,020,358 discloses the Apremilast compound and the process for its preparation.

Various oral dosage forms of Apremilast are known, U.S. Pat. Nos. 7,427,638, 9,351,957, US patent application no. 20130164376, US patent application no. 20150306226 disclose different oral dosage forms.

However oral dosage forms have sufficient evidential side effects like diarrhoea, nausea and headache. Besides these common side effects, other gastrointestinal disorders are also observed with oral Apremilast. General, oral route is a suboptimal method to treat diseases of the skin. Particularly the systemic administration of the drug (via the oral route or otherwise) carries risk of side effect in tissues unconnected with such conditions.

To overcome oral drugs first pass metabolism by the liver, and evident side effects, topical administration would frequently be the route of choice, if it could be achieved. PCT publication no. WO2016198469 discloses topical formulations of water-free emulsions for penetration of alpha-hydroxy acids. PCT publication no. WO2016198472 discloses topical delivery system comprising of a water-free emulsion of a discontinuous polar phase in a continuous lipid phase that will deliver certain compounds to the skin preferably certain amino-guanidines dissolved in the polar phase, in combination with a second topically active drug.

WO 2018/138737 discloses topical compositions of Apremilast with specific alcoholic, aqueous carriers and excipients. WO 2017/216738 discloses 0.1 to 5% topical composition of Apremilast free from alpha-hydroxy acids, with specific ratio of penetration enhancer/solubilizer. CN105168136 discloses an Apremilast preparation with cholesterol and phospholipids as essential ingredients. WO 2017/168433 discloses an Apremilast topical composition with specific drug to carrier ratio. CN108283620A discloses a topical composition of Phosphodiesterase inhibitor with DMSO and carrier.

A drawback of above prior art compositions and routine conventional topical formulations is that their active component acts for a short duration of time, thereby requiring frequent re-application which leads to a negative impact on treatment compliance and quality of life of the patient. Also some topical formulation can often irritate normal skin, may be time consuming and awkward to apply, also can stain clothing, and/or have an objectionable odour, so cannot be used for longer periods. Consequently it is sometimes difficult for patients to maintain regular applications of these medications. Moreover abrupt withdrawal of Apremilast topical agent may cause an aggressive recurrence of the condition or severe impact on overall patient health.

Despite the presence of various topical compositions there is need in the art for a stable and highly effective, convenient composition which can provide controlled/sustained release of the drug and is consistent and non-greasy on skin over conventional topical compositions. Also the key aspect for topical composition is penetration or diffusion into the skin layers for better efficacy. However due to the insolubility of Apremilast itself in water and minimal solubility in polar solvents, it is challenging to formulate a proper topical composition of Apremilast having better penetrating capabilities and providing an effective concentration of Apremilast at the site of action for a longer period of time to give enhanced therapeutic results. Hence, there remains an unmet need to develop suitable formulation like topical creams, ointments, gels and dispersions which can give better penetration and efficacy with reduced dosage and frequency of administration.

The inventors of the present invention found that topical composition of Apremilast with specific combination of lipophilic components, significantly achieve an enhanced skin penetration, thus better efficacy with site specific drug delivery and provides a much better treatment approach. The topical compositions of the invention can be used for psoriasis and related skin conditions.

### SUMMARY OF THE INVENTION

The present invention provides a topical composition of Apremilast with at least one lipophilic excipient.

A further object of the invention is to provide a topical composition of Apremilast with enhanced skin penetration and feel-good consistency.

Another object of present invention is to provide a longer acting controlled release topical composition of Apremilast.

A further object of the present invention is to provide a topical pharmaceutical composition suitable for topical application, comprising Apremilast or its pharmaceutically acceptable salt in therapeutically effective amounts in combination with pharmaceutically acceptable excipients.

Yet another object of present invention is to provide a topical composition of Apremilast with specific quantity of at least one lipophilic vehicle, at least one emulsifier and a pH adjusting agent to maintain a pH in the suitable range for better stability of the formulation.

A further object of the invention is to provide topical compositions like cream, gel, ointment, lotion, foam, dispersion, paste or spray which are characteristically long acting, and provide enhanced drug delivery and skin permeation. In another embodiment, the application provides processes for preparing pharmaceutically stable topical compositions comprising Apremilast

An another object of the present invention is to provide a topical cream or ointment of Apremilast with specific quantity of at least one lipophilic vehicle, at least one emulsifier and a pH adjusting agent to maintain a pH in the suitable range for better stability of the formulation and to provide a longer acting controlled release topical composition of Apremilast.

A yet another object of the present invention is to provide a topical gel of Apremilast with specific quantity of at least one lipophilic vehicle, at least one emulsifier and a pH adjusting agent to maintain a pH in the suitable range for better stability of the formulation and to provide a longer acting controlled release topical composition of Apremilast.

Another object of the present invention is to provide a process for preparation of a lipophilic Apremilast topical composition.

In yet another object the present invention relates to a stable topical composition of Apremilast for treatment or amelioration of diseases such as psoriasis or psoriatic arthritis, dermatosis, eczema, rosacea, acne, contact and atopic dermatitis, pruritus, inflammation and other associated skin conditions.

### DETAILED DESCRIPTION

The inventors of the present invention are consistently working towards approaches for eradication of psoriasis and possible treatment methodologies by developing various compositions for better and effective remedy. In line with the ongoing research the inventors have surprisingly found out that there is significant enhanced penetration of the drug with lipophilic vehicles in particular concentration ranges and combination, which was further evaluated with other components or excipients in order to obtain better acting and effective compositions. The topical composition in the present invention is one of the successful outcomes of the ongoing research.

The present disclosure is herein described in detail with reference to embodiments, which form a part here. Other embodiments may be used and/or other changes may be made without departing from the spirit or scope of the present disclosure. The illustrative embodiments described in the detailed description are not meant to be limiting of the subject matter presented here.

The present invention pertains to a stable topical formulation of Apremilast with at least one lipophilic vehicle for topical treatment of skin conditions in humans. In combination therewith is a plurality of excipients in specific quantity and combinations, for both solubilizing the active agent and forming a long acting, better penetrating consistent topical composition.

In another embodiment, the present invention provides a topical cream, ointment or gel composition of Apremilast with at least one lipophilic excipient and having enhanced skin penetration, better patient compliance and consistency.

In yet another preferred embodiment, the present invention provides a topical composition of Apremilast with specific quantity of at least on lipophilic vehicle, at least one emulsifier and a pH adjusting agent to maintain a pH in the suitable range for better stability and shelf life of the formulation, and to provide a longer acting controlled release topical composition of Apremilast.

In another embodiment the present invention relates to a stable topical composition of Apremilast for treatment of psoriasis or psoriatic arthritis.

In yet another embodiment the present invention also provides a method for effectively treating psoriatic symptoms and other related skin disorders using a lipophilic composition of Apremilast.

Further it has been found that pharmaceutical compositions in the form of topical creams, ointments, lotions, dispersions and gels as prepared according to present invention provide desired pharmacological actions and fewer side effects, along with better patient compliance.

In one embodiment of the invention, Apremilast is provided in the pharmaceutical compositions in the form of topical cream, ointment, lotion, gel or topical ophthalmic gel.

In another embodiment of the invention, Apremilast is provided as a topical pharmaceutical composition with one or more lipophilic vehicles, or combinations thereof.

In another preferred embodiment, the inventors of the present invention have developed the topical pharmaceutical composition of Apremilast with enhanced diffusion rate and penetration.

In an embodiment of the invention, topical composition may comprise excipients that are hydrophilic and/or hydrophobic in nature.

In a most preferred embodiment of the invention, the topical composition comprises of Apremilast or its pharmaceutically acceptable salt and a lipophilic agent or a mixture of two or more lipophilic agents in suitable ratio, to give maximum average flux or diffusion of the drug through the skin layers.

In another preferred embodiment of the invention, the topical composition comprises of Apremilast or its pharmaceutically acceptable salt and a mixture of first lipophilic agent and a second lipophilic agent, wherein the first and second lipophilic agents could be same or different lipophilic agent in suitable ratio, to give maximum average flux or diffusion of the drug through the skin layers.

Unless otherwise recited or required by the context, percent and “%” refer to percent by weight.

Topical pharmaceutical compositions of the invention include Apremilast in the concentration of 1% to 10% by weight, preferably 1-5% w/w.

Topical pharmaceutical compositions of the invention include Apremilast and lipophilic vehicle in an amount of 1-10% w/w and 2-50% w/w respectively or in the ratio of 1:2 to 10:50.

Topical pharmaceutical compositions of the invention have a pH in the physiological range between 3 to 8 or 4 to 7 or 4.5 to 6.5.

In yet another preferred embodiment the ratio of drug to the lipohilic agent in the composition is ranging from 1:100 to 100:1 or 1:50 to 50:1 or 1:5 to 10:50.

In yet another embodiment the lipophilic agent in the composition can be a single lipophilic agent or a combination of lipophilic agents, combined in such a concentration to impart maximum drug diffusion through the skin.

In an embodiment, the ratio of Apremilast to lipophilic agent is from about 1:2 to about 10:50. In another embodiment, the ratio of Apremilast to lipophilic agent is from about 1:5 to about 5:50. In yet another embodiment, the ratio of Apremilast to lipophilic agent is about 1:10 to 5:30. In a further embodiment, the ratio of Apremilast to lipophilic agent is about 1:8, 1:10, 2:12, 3:14, 4:16 or 5:20. In yet another embodiment, the ratio of Apremilast to lipophilic agent is about 1:5.

In an embodiment, the ratio of drug to lipophilic agents is drug:first lipophilic agent:second lipophilic agent, which is 2:10:10 or 4:10:10 or 2:10:12 or 4:10:12. In a further embodiment the drug and lipohilic agent is present in a specific ratio with a suitable emulsifier in a ratio of 2:10:12 or 5:10:12. Further the first and second lipophilic agent could be same or different lipophilic agents.

In another preferred embodiment, the lipophilic topical composition of the invention comprises Apremilast 1-10% w/w, 1^{st} lipophilic agent 2-50% w/w, 2^{nd} lipophilic agent 2-50% w/w, emulsifier 2-30%, co-emulsifier 2-25% w/w, solvent/carrier 2-40% w/w, preservative 0.05-5% w/w, carrier 10-80% w/w and suitable pH adjusting agent or buffering agent.

In yet another preferred embodiment, the lipophilic topical composition of the invention comprises Apremilast 1-10% w/w, at least one lipophilic agent 2-50%, emulsifier 2-30%, solvent/carrier 2-40% w/w, preservative 0.05-5% w/w, carrier 10-80% w/w and suitable pH adjusting agent or buffering agent.

In another embodiment, the lipophilic topical composition comprises of Apremilast in an amount from about 1-8% by weight of the composition; the lipophilic vehicle in an amount of 6-25% by weight of the composition; the emulsifier in an amount of 2-18% by weight of the composition; and the pH adjusting agent is in an amount of 0.05-0.5% by weight of the composition.

In yet another embodiment the present invention provides a method for effectively treating psoriatic symptoms and other related skin disorders by administering a lipophilic composition of Apremilast comprising of Apremilast 1-10% w/w, at least one lipophilic agent 2-50%, emulsifier 2-30%, solvent/carrier 2-40% w/w, preservative 0.05-5% w/w, carrier 10-80% w/w and suitable pH adjusting agent or buffering agent.

In yet another embodiment of the invention, topical composition also comprises excipients to provide better feel to the skin, no greasiness or feel of any gritty particles and lower irritation to the skin.

In one of the embodiment of the invention, topical pharmaceutical composition may optionally comprise of suitable preservative or fragrance.

In one preferred embodiment, the invention is directed to a topical composition for treating psoriasis comprising a therapeutically effective amount of Apremilast, a lipophilic vehicle and a pharmaceutically acceptable carrier.

A “therapeutically effective amount” is an amount necessary to palliate at least one symptom of psoriasis. For example, a therapeutically effective amount is sufficient to treat (i.e. alleviate or reduce) at least one of: itching/scratching, redness, inflammation, cracking, scaling, bleeding, etc. Preferably, the therapeutically effective amount of Apremilast comprises between 1 to 10% by weight of the composition, more preferably 1 to 5% w/w.

In an embodiment of the invention, topical composition of present invention has excipients that help deep and effective penetration of Apremilast in to the skin and provide ease of application, spreadability and cleaning.

In another preferred embodiment the process for preparation of the lipophilic topical composition characteristically involves drug addition to non-aqueous phase or the drug is added only to the oil phase. Also the addition of the drug can be done before or after the formation of dispersion or cream.

Non-Limiting Lists of the Excipients that can be Used in the Composition are:

Preferred non-limiting examples of lipophilic agents useful in the present composition include small and medium chain triglycerides like PG monocaprylocaprate, PG dicaprylocaprate (Labrafac PG), glycerol tricaprylocaprate (Labrafac Lipophile WL1349), other lipophilic agents may include petrolatum, red petrolatum, white petrolatum, liquid petrolatum, semi-solid petrolatum, light mineral oil, heavy mineral oil, white mineral oil, mineral oil alcohols, C₇-C₄₀ branched chain hydrocarbons, C₁₀-C₃₀ alcohol esters of C₁₀-C₃₀ carboxylic acids, C₁₀-C₃₀ alcohol esters of C₁₀-C₃₀ dicarboxylic acids, monoglycerides of C₁₀-C₃₀ carboxylic acids, diglycerides of C₁₀-C₃₀ carboxylic acids, triglycerides of C₁₀-C₃₀ carboxylic acids, ethylene glycol monoesters of C₁₀-C₃₀ carboxylic acids, ethylene glycol diesters of C₁₀-C₃₀ carboxylic acids, propylene glycol monoesters of C₁₀-C₃₀ carboxylic acids, propylene glycol diesters of C₁₀-C₃₀ carboxylic acids, C₁₀-C₃₀ carboxylic acid monoesters and polyesters of sugars, vegetable oils, hydrogenated vegetable oils, olive oil, hydrogenated olive oil, Shea butter, polypropylene glycols, polypropylene glycol C₄-C₂₀ alkyl ethers, di C₈-C₃₀ alkyl ethers, synthetic hydrocarbons, derivatives thereof, and mixtures thereof.

Additionally preferred, non-limiting examples of such other lipophilic agents include ethylene glycol distearate, cetyl palmitate, myristyl myristate, stearyl stearate, cetyl stearate, behenyl behenate, caprylic/capric triglyceride, PEG-6 caprylic/capric triglyceride, PEG-8 caprylic/capric triglyceride, derivatives thereof, and mixtures thereof.

In addition to the above agents, the present preferred compositions can optionally contain an additional lipophilic agent as an emollient. Many of the above-mentioned agents can alternatively be present in the instant compositions as an emollient in this regard. It would be readily apparent to a person of ordinary skill in the art exactly which of these components exhibit utility as an emollient.

Further the composition may comprise pharmaceutically acceptable carriers like water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, nano- and micro-particulate carriers, and/or titanium dioxide. More preferably, the pharmaceutically acceptable carrier comprises dimethyl sulfoxide (DMSO), glycerol, propylene glycol, petrolatum water, and one or more pharmaceutically acceptable penetration enhancer (absorption promoter and/or accelerants).

Typically, the topical compositions of the invention comprise skin penetration enhancers, pharmaceutical surfactants and solubility enhancers, oil phase components, aqueous phase components, emulsifiers, moisturizers, antioxidants, vitamins, lubricants, preservatives, stabilizers, buffers and other ingredients. Also the components/excipients as embodied in the present composition may have more than one application.

Skin penetration enhancers reversibly decrease the barrier resistance of the skin, which increases the amount of Apremilast absorbed. Preferably, skin penetration enhancers include, but are not limited to, lipophilic PGs (Propylene Glycols), sulfoxides (e.g. DMSO), azones (e.g. laurocapram), pyrrolidones (e.g., 2-pyrrolidone), alcohols and alkanols (e.g., ethanol, decanol, etc.), oleic acid (and derivatives thereof), glycols (e.g., propylene glycol), dimethylformamide (DMF), dimethylacetamide (DMAC), fatty alcohols (e.g., lauryl alcohol), fatty acid esters, fatty acids, fatty alcohol ethers (e.g., EO-2-oleyl ether), terpenes, and biologics (e.g., lecithin).

Pharmaceutical surfactants or solubility enhancers include, but are not limited to, lauryl alcohol, polyoxyethylene ether, polyoxyethylene glycerol monostearate, stearic acid ester oxygen poly hydrocarbon, vitamin E succinate polyethylene glycol ester, sorbitan esters, polyoxyethylene castor oil, polyoxyethylene hydrogenated castor oil, poloxamer, organic esters (e.g. ethylene acetate), and poly hill dinitrate 80 (i.e. Tween 80 or its mixture). In a preferred embodiment, the pharmaceutical surfactants or solubility enhancers include DMSO, polyvinylpyrrolidones, stearic acid hydrocarbon oxygen Poly (40) ester, lauryl alcohol polyoxyethylene (23) ether, vitamin E succinate polyethylene glycol ester, ethylene acetate, and polyoxyethylene (40) hydrogenated castor oil (and its mixtures, i.e. polyoxy (40) stearate). Still, in a more preferred embodiment the pharmaceutical surfactants or solubility enhancers include sodium lauryl sulphate and sorbitan esters.

Suitable oily phase may include, but are not limited to, glyceryl monoacetate, glycerol diacetate, glyceryl triacetate, stearic acid, soybean oil, corn oil, peanut oil, palmitic acid, palm oil, sunflower oil, olive oil, coconut oil, sesame oil, cotton seed oil, rapeseed oil, oleic acid, medium-chain triglycerides, single-decane triglyceride, animal fat (e.g., lanolin), mineral oils, paraffin, beeswax, petrolatum, hydrocarbons, vaseline, and mixtures thereof.

Aqueous phase components include, but are not limited to, de-ionized water, glycerol gelatin, cellulose derivatives (e.g., microcrystalline cellulose (Avicel PH 101)), and polyethylene glycol (PEG 300 to PEG 6000), and mixtures thereof.

Emulsifiers include, but are not limited to Emulcire 61 WL 1349, Gelot 64, oleyl alcohol, polyoxyethylene oleyl ether, PEG-40 stearate, ceteareth-12, ceteareth-20, ceteareth-30, glyceryl stearate, PEG-100 stearate, methyl myristate, isopropyl myristate, Arlacel 165, glyceryl stearate, PEG-100 stearate, steareth-2 and steareth-20, dimethicone copolyol, Polysorbate 20 (Tween 20), cetyl esters wax, Polysorbate 40 (Tween 40), Polysorbate 60 (Tween 60), Polysorbate 80 (Tween 80), lauramide DEA, cocamide DEA, and cocamide MEA, Phospholipid PTC, alginate, carrageenan, Glucate DO, methylcellulose, polyvinyl alcohol, Carbopol and Carbomer. Preferably, emulsifiers are selected from the group consisting of cetostearyl alcohol, stearic acid, magnesium stearate, sodium lauryl sulfate, triethanolamine, and magnesium aluminum silicate.

Any other emulsifiers known to those of skill in the art as useful in the formation of topical compositions are further contemplated herein.

Moisturizers include, but are not limited to, glycerol, pentylene glycol, butylene glycol, polyethylene glycol, sodium pyrrolidone carboxylate, alpha-hydroxy acids, beta-hydroxy acids, polyhydric alcohols, ethoxylated and propoxylated polyols, polyols, polysaccharides, panthenol, hexylene glycol, propylene glycol, dipropylene glycol, sorbitol, derivatives thereof, and mixtures thereof.

Antioxidants include, but are not limited to, water soluble antioxidants, lipid-soluble antioxidants, vitamin C, vitamin C palmitate, propyl gallate, vitamin E (tocopherol), tert-butyl ether-hydroxybenzoate fennel, 2,6 di-tert-butyl-p-cresol, BHA, BHT, or mixtures of one or more antioxidants.

Lubricants include, but are not limited to, urea, magnesium stearate, sodium lauryl sulfate, polyethylene glycol, and silica gel powder.

Preservatives include, but are not limited to, chloro-m-cresol, citric acid, disodium edetate, ethoxylated alcohol, glycerin, 1,2,6-hexanetriol, methylparaben, parabens, potassium, sorbate, propyl gallate, propylene glycol, propyl paraben, sodium bisulfate, sodium citrate, butyl paraben, sodium metabisulfite, chlorocresol, sorbic acid, tannic acid, zinc stearate, butylated hydroxytoluene, butylated hydroxyanisole, benzoic acid, salicylic acid, propyl paraben, dichlorobenzyl alcohol, formaldehyde, alpha-tocopherol, sodium ascorbate, ascorbic acid, ascorbyl palmitate phenol, m-cresol, bisphenol, cetrimide, benzalkonium chloride, sorbic acid, phenoxyethanol, and benzoyl peroxide. Preferably, preservatives are selected from the group consisting of hydroxylethyl benzene, hydroxylmethyl benzene, phenoxyethanol, chlorocresol, propyl paraben, and methyl paraben.

The presently preferred compositions may further contain a pH modifier. Preferably, the presently preferred compositions can comprise about 0.01% to about 3% by weight of a pH modifier. Preferred non-limiting examples of neutralizing pH modifiers that can optionally be included in these compositions include inorganic hydroxides, inorganic oxides, inorganic salts of weak acids, derivatives thereof, and mixtures thereof.

Preferred non-limiting examples of inorganic hydroxides useful in this regard include ammonium hydroxide, alkali metal hydroxide, alkaline earth metal hydroxides, derivatives thereof, and mixtures thereof.

Preferred inorganic hydroxides useful in this regard include ammonium hydroxide, monovalent alkali metal hydroxides such as sodium hydroxide and potassium hydroxide, divalent alkali earth metal hydroxides such as calcium hydroxide and magnesium hydroxide, derivatives thereof, and mixtures thereof.

Preferred non-limiting examples of inorganic oxides useful in this regard include magnesium oxide, calcium oxide, derivatives thereof, and mixtures thereof.

Preferred, non-limiting examples of inorganic salts of weak acids useful in this regard include ammonium phosphate (dibasic), alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic), alkaline earth metal salts of weak acids such as magnesium phosphate and calcium phosphate, derivatives thereof, and mixtures thereof.

In an embodiment of the invention, topical composition of present invention has pH adjusting agents that help in adjusting the pH of the composition between pH of about 3 to about 8 to provide a stable and non-irritating composition. Such agents include many pharmaceutically acceptable acids, bases and buffers. Suitable acids may include one or more of hydrochloric acid, phosphoric acid and lactic acid. Suitable bases may include one or more of diethanolamine, triethanolamine, triethylamine and sodium hydroxide. Suitable buffers may include phosphates, such as monobasic sodium phosphate, dibasic sodium phosphate, lactates and citrates.

### Definitions

As used here, the following terms have the following definitions:

“Active Pharmaceutical Ingredients (APIs)” refer to chemical compounds that induce a desired effect, and include agents that are therapeutically or prophylactically effective.

“Permeation enhancement” refers to an increase in the permeability of the skin or mucosal tissue to the selected active pharmaceutical ingredients.

“Topical administration” refers to delivery of a topical drug or active pharmaceutical ingredient to the skin or mucosa, thus providing a local effect.

“Transdermal pharmaceutical composition” refers to topical medications that may be used in different application forms, such as for example creams, lotion patches, pastes, gels, and the like, and which release one or more active drugs through the stratum corneum at a predetermined rate over a defined period of time to a defined site of application.

“Treating” or “Treatment” refers to reduction in severity and/or frequency of symptoms, elimination of symptoms and/or underlying cause, prevention of the occurrence of symptoms and/or their underlying cause, and improvement or remediation of damage.

The term “pharmaceutically acceptable” means approved by a regulatory agency of the Federal or state government or listed in the U.S. Pharmacopeia or other generally recognized pharmacopeia for use in animals, and more particularly in humans.

The term “Lipophilic agent” refers to vehicle, carrier, diluent, adjuvant, excipient, penetration enhancer, or solubilizer with which an active ingredient is administered. Such pharmaceutical agents can be lipophilic or hydrophobic components such as lipids and oils, including those of petroleum, animal, vegetable or synthetic origin. Preferably, the lipophilic agent comprises hydrophobic excipients commonly used in topically applied formulations.

The term “carrier” refers to a diluent, adjuvant, excipient, penetration enhancer, or vehicle with which an active ingredient is administered.

### EXAMPLES

While the invention will now be described in connection with certain preferred embodiments in the following examples so that aspects thereof may be more fully understood and appreciated, it is not intended to limit the invention of these particular embodiments. On the contrary, it is intended to cover all alternatives, modifications and equivalents as may be included within the scope of the invention as defined by the appended claims.

Thus, the following examples which include preferred embodiments will serve to illustrate the practice of this invention, it being understood that the particulars shown are by way of example and for purposes of illustrative discussion of preferred embodiments of the present invention only and are presented in the cause of proving what is believed to be the most useful and readily understood description of formulation procedures as well as of the principles and conceptual aspects of the invention. It will be evident to those skilled in the art that the invention is not limited to the details of the following illustrative examples and that the present invention may be embodied in other specific forms without departing from the essential attributes thereof, and it is therefore desired that the present embodiments and examples be considered in all respects as illustrative and not restrictive, reference being made to the appended claims, rather than to the foregoing description, and all changes which come within the meaning and range of equivalency of the claims are therefore intended to be embraced therein.

### Example 1: Apremilast Topical Cream

| Component | % w/w |
|---|---|
| Apremilast | 1-10 |
| Labrafac PG | 2-50 |
| Labrafac Liphophile WL 1349 | 2-50 |
| Emulcire 61 WL 2659 | 2-30 |
| Gelot 64 | 2-25 |
| DMSO | 2-40 |
| Methyl paraben | 0.05-5 |
| propyl paraben | 0.01-5 |
| Glycerine | 10-80 |
| 10% NaOH Solution | q.s. |
| Triethanolamine | q.s. |
| Total | 100 |

Brief Manufacturing Process:
1. Apremilast was dissolved in a suitable carrier.
2. Lipophilic agents like Labrafac PG and Labrafac Lipophile WL 1349 were mixed together and heated to about 70-75° C.
3. The preservatives were added to step 2 lipophilic mixture (oil phase) under stirring.
4. After solubilization of preservatives, suitable emulsifiers were added under stirring to the step 3 mixture, the temperature of the oil phase was maintained at 70-75° C.
5. After solubilization of emulsifiers, the temperature of the mixture was decreased to about 65-70° C.
6. Step 1 drug solution was then added to Step 5 oil phase under stirring and temperature of the oil phase was maintained at about 65-70° C.
7. Thereafter a suitable carrier was heated to about 65-70° C. and added drop wise to the step 6 oil phase under stirring. The stirring was continued till formation of a dispersion.
8. The pH of the cream was adjusted to a pH of 4.0-7.5 using buffering/pH adjusting agent.

Observations:
i. The resulting cream was found to be of adequate consistency;
ii. The formulation was free of gritty particles and greasiness;
iii. The pH of the formulation was found to be in range of 4.0-7.5;

### Example 2: Apremilast Topical Cream

| Component | % w/w | mg/gm |
|---|---|---|
| Apremilast | 4 | 40 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Purified water | 50.84 | 508.4 |
| 10% NaOH Solution | q.s. | q.s |
| Total | 100 | 1000 |

Brief Manufacturing Process-I:
1. Apremilast was dissolved in DMSO.
2. Labrafac PG and Labrafac Lipophile WL 1349 were mixed together and heated to 70-75° C.
3. Preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 50-55° C.
6. The step 1 drug solution was added to step 5 oil phase under stirring and temperature of the oil phase was maintained at 50-55° C.
7. Thereafter purified water was heated to 50-55° C. and added drop wise to the step 6 oil phase under stirring. The stirring was continued till formation of a cream.
8. The pH of the cream of step 7 was adjusted to 6.5 using 10% w/w NaOH solution.

Observations:
i. The resulting cream was unstable, as API was precipitated after water addition;
ii. The consistency and greasiness of the cream was adequate.

Inference: The precipitation of formulation was concluded to be due to the exposure of API to purified water, thereby it was proposed to change the order of adding drug into the formulation.

Brief Manufacturing Process-II:
1. Apremilast was dissolved in DMSO.
2. Labrafac PG and Labrafac Lipophile WL 1349 were mixed together and heated to 70-75° C.
3. Preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 50-55° C.
6. Thereafter purified water was heated to 50-55° C. and added drop wise to the step 5 oil phase under stirring. The stirring was continued till formation of a cream.
7. After the formation of the cream, the step 1 drug solution was added drop-wise to the step 6 cream and the pH of the cream of was adjusted to 6.5 using 10% w/w NaOH solution.

Observations:
i. The resulting cream was unstable, as API was precipitated after addition of drug;
ii. The consistency and greasiness of the cream was adequate; and
iii. Gritty particles observed due to API precipitation.

Inference: The precipitation was concluded to be due to the presence of purified water, thereby it was proposed to replace water from the formulation with other suitable aqueous/non-aqueous solvents.

### Example 3: Apremilast Topical Cream

| Component | % w/w | mg/gm |
|---|---|---|
| Apremilast | 4 | 40 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Glycerine | 50.84 | 508.4 |
| 10% NaOH Solution | q.s. | q.s. |
| Total | 100 | 1000 |

Brief Manufacturing Process-I:
1. Apremilast was dissolved in DMSO.
2. Labrafac PG and Labrafac Lipophile WL 1349 were mixed together and heated to 70-75° C.
3. Preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 50-55° C.
6. Thereafter Glycerine was heated to 50-55° C. and added drop wise to the step 5 oil phase under stirring. The stirring was continued till formation of a cream.
7. After cream formation, step 1 drug solution was the added to the step 6 cream under stirring.
8. The pH of the cream of step 7 was adjusted to 6.5 using 10% w/w NaOH solution.

Observations:
i. The resulting cream was found to be of adequate consistency, greasiness and viscosity.
ii. Gritty particles were not observed; and
iii. On storage phase separation was observed.

Inference: The phase separation was concluded to be due to the variation in the rotation speed and process of addition of API solution.

Brief Manufacturing Process-II:
1. Apremilast was dissolved in DMSO.
2. Labrafac PG and Labrafac Lipophile WL 1349 were mixed together and heated to 70-75° C.
3. The preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 65-70° C.
6. Step 1 drug solution was then added to Step 5 oil phase under stirring and temperature of the oil phase was maintained at 65-70° C.
7. Thereafter Glycerine was heated to 65-70° C. and added drop wise to the step 6 oil phase under stirring. The stirring was continued till formation of a cream.
8. The pH of the resulting cream from step 7 was adjusted to 6.5 using 10% w/w NaOH solution.

Observations:
i. The resulting cream was found to be of adequate consistency, free of gritty particles and greasiness;
ii. The cream was found to have good smoothness and appearance, however while adjusting the pH with 10% NaOH solution, the appearance and smoothness decreased upto some extent (minor) with no changes in the consistency, greasiness and viscosity.

Inference: The decrease in appearance and smoothness was concluded to be due to the variation in the presence of water in 10% w/w NaOH solution, thus it was proposed to use organic solution like triethanolamine, tri-ethylamine, etc.

### Example 4: Apremilast Topical Cream

| Component | % w/w | mg/gm |
|---|---|---|
| Apremilast | 4 | 40 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Glycerine | 50.84 | 508.4 |
| Triethanolamine | q.s. | q.s. |
| Total | 100 | 1000 |

Brief Manufacturing Process:
1. Apremilast was dissolved in DMSO.
2. Labrafac PG and Labrafac Lipophile WL 1349 were mixed together and heated to 70-75° C.
3. The preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 65-70° C.
6. Step 1 drug solution was then added to Step 5 oil phase under stirring and temperature of the oil phase was maintained at 65-70° C.
7. Thereafter Glycerine was heated to 65-70° C. and added drop wise to the step 6 oil phase under stirring. The stirring was continued till formation of a cream.
8. The pH of the resulting cream from step 7 was adjusted to 6.5 using triethanolamine.

Observations:

The resulting cream was found to be of smooth in appearance, has adequate consistency, free of gritty particles and greasiness.

Inference: The use of organic solution does not have much impact on the diffusion rate thus the use of 10% w/w NaOH solution or organic solution like triethanolamine, triethylamine, etc., for adjusting pH do not have any significant difference on efficacy of formulation.

### Example 5: Apremilast Topical Cream

| Component | % w/w | mg/gm |
|---|---|---|
| Apremilast | 2 | 20 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Glycerine | 52.84 | 528.4 |
| 10% NaOH solution | q.s. | q.s. |
| Total | 100 | 1000 |

Brief Manufacturing Process:
1. Apremilast was dissolved in DMSO.
2. Labrafac PG and Labrafac Lipophile WL 1349 were mixed together and heated to 70-75° C.
3. Preservatives Methyl paraben and Propyl paraben were added to step 2 lipophilic mixture (oil phase) under stirring.
4. After solubilization of methyl and propyl paraben, Emulcire 61 WL 2659 and Gelot 64 were added under stirring to the step 3 mixture, the temperature of the oil phase was maintained at 70-75° C.
5. After solubilization of Emulcire 61 WL 2659 and Gelot 64, the temperature of the mixture was decreased to 65-70° C.
6. Step 1 drug solution was then added to Step 5 oil phase under stirring and temperature of the oil phase was maintained at 65-70° C.
7. Thereafter Glycerine was heated to 65-70° C. and added drop wise to the step 6 oil phase under stirring. The stirring was continued till formation of a cream.
8. The pH of the resulting cream from step 7 was adjusted to 6.5 using 10% NaOH solution. (Also similar samples with pH 3.9 and 6.9 prepared for evaluation study)

Observations:

The resulting cream was found to be of adequate consistency, free of gritty particles and greasiness.

### Example 6: Apremilast Topical Composition

| Component | % w/w | mg/gm |
|---|---|---|
| Apremilast | 6 | 60 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Glycerine | 48.84 | 488.4 |
| 10% NaOH solution | q.s. | q.s. |
| Total | 100 | 1000 |

Composition prepared using the process same as Example-5.

### Example 7: Apremilast Topical Composition

| Component | % w/w | mg/gm |
|---|---|---|
| Apremilast | 8 | 80 |
| Labrafac PG | 10 | 100 |
| Labrafac Liphophile WL 1349 | 12 | 120 |
| Emulcire 61 WL 2659 | 9 | 90 |
| Gelot 64 | 3.6 | 36 |
| DMSO | 10 | 100 |
| Methyl paraben | 0.5 | 5 |
| propyl paraben | 0.06 | 0.6 |
| Glycerine | 46.84 | 468.4 |
| 10% NaOH solution | q.s. | q.s. |
| Total | 100 | 1000 |

Composition prepared using the process same as Example-5.

### Example 8: Comparative Diffusion Study

To evaluate the efficacy of the present invention lipophilic composition the applicant performed diffusion study, wherein a comparison of the claimed lipophilic composition as prepared in Examples 3, 4 & 5 were made with prior art aqueous topical Apremilast compositions prepared as per the procedures disclosed in WO2017168433 (herein after 433′). Also impact of pH on diffusion data was analysed as shown in Table-1, below:

TABLE 1
Diffusion study of different formulation:

| | Dosage Form | | | | | |
|---|---|---|---|---|---|---|
| | | | Topical cream (Lipophilic) | | | |
| | Topical Gel (Aqueous) | | Ex-3 | Ex-5 | Ex-5 | Ex-4 |
| Example | 433′ | 433′ | (pH 3.7) | (pH 3.9) | (pH 6.9) | (pH 6.8) |
| Strength | 2% w/w | 4% w/w | 4% w/w | 2% w/w | 2% w/w | 4% w/w |
| No. of Samples | 2 | 2 | 3 | 2 | 2 | 2 |
| Avg. Flux | 0.98 | 1.45 | 11.69 | 17.59 | 12.44 | 8.38 |
| (μg/sq. cm/hr) | | | | | | |
| Avg. cumulative DR | 176.5 | 196.5 | 1550 | 1407.5 | 1192.35 | 1497.25 |
| in μg (after 48 hrs) | | | | | | |
| Avg % DR | 2.94 | 1.59 | 12.92 | 23.41 | 19.27 | 12.22 |
| (after 48 hrs) | | | | | | |

(Abbreviation Avg. = average, % = percentage, DR = drug release)

From the above study it was concluded that the present application lipophilic composition has a very high average flux (diffusion rate) and drug release, thus it is much superior and effective in penetrating the skin layers over prior topical compositions of Apremilast. Further pH study revealed that the composition is stable and effective over wide range of pH ranging from 3-8 Thus the proposed formulation was found to be stable, free from gritty particles and greasiness, consistent throughout the shelf life and has a very high diffusion rate thus better drug release profile.

### Example 9: Apremilast Topical Composition

| Component | % w/w | Mg/gm |
|---|---|---|
| Apremilast | 5 | 50 |
| Labrafac PG | 20 | 200 |
| Labrafac Liphophile WL 1349 | 18 | 180 |
| Emulcire 61 WL 2659 | 15 | 150 |
| Gelot 64 | 8.6 | 86 |
| DMSO | 10 | 100 |
| Methyl paraben | 0.5 | 5 |
| propyl paraben | 0.06 | 0.6 |
| Glycerine | 22.84 | 228.4 |
| 10% NaOH solution | q.s. | q.s. |
| Total | 100 | 1000 |

Composition prepared using the process same as Example-5.

### Example 10: Apremilast Topical Composition

| Component | % w/w | mg/gm |
|---|---|---|
| Apremilast | 1 | 10 |
| Labrafac PG | 25 | 250 |
| Labrafac Liphophile WL 1349 | 8 | 80 |
| Emulcire 61 WL 2659 | 12 | 120 |
| Gelot 64 | 5.6 | 56 |
| DMSO | 15 | 150 |
| Methyl paraben | 0.5 | 5 |
| propyl paraben | 0.06 | 0.6 |
| Glycerine | 32.84 | 328.4 |
| 10% NaOH solution | q.s. | q.s. |
| Total | 100 | 1000 |

Composition prepared using the process same as Example-5.

1. A lipophilic topical composition of Apremilast comprising Apremilast or its pharmaceutically acceptable salt with at least one lipophilic agent and pharmaceutically acceptable excipients.

2. The lipophilic topical composition as claimed in claim 1, wherein the lipophilic agent is selected from the group comprising of small and medium chain triglycerides.

3. The lipophilic topical composition as claimed in claim 1, wherein the lipophilic agent is selected from the group comprising of PG monocaprylocaprate, PG dicaprylocaprate (Labrafac PG), glycerol tricaprylocaprate (Labrafac Lipophile WL1349), petrolatum, red petrolatum, white petrolatum, liquid petrolatum, semi-solid petrolatum, light mineral oil, heavy mineral oil, white mineral oil, mineral oil alcohols, C₇-C₄₀ branched chain hydrocarbons, C₁₀-C₃₀ alcohol esters of C₁₀-C₃₀ carboxylic acids, C₁₀-C₃₀ alcohol esters of C₁₀-C₃₀ dicarboxylic acids, monoglycerides of C₁₀-C₃₀ carboxylic acids, diglycerides of C₁₀-C₃₀ carboxylic acids, triglycerides of C₁₀-C₃₀ carboxylic acids, ethylene glycol monoesters of C₁₀-C₃₀ carboxylic acids, ethylene glycol diesters of C₁₀-C₃₀ carboxylic acids, propylene glycol monoesters of C₁₀-C₃₀ carboxylic acids, propylene glycol diesters of C₁₀-C₃₀ carboxylic acids, C₁₀-C₃₀ carboxylic acid monoesters and polyesters of sugars, vegetable oils, hydrogenated vegetable oils, olive oil, hydrogenated olive oil, Shea butter, polypropylene glycols, polypropylene glycol C₄-C₂₀ alkyl ethers, di C₈-C₃₀ alkyl ethers, synthetic hydrocarbons, calamine, derivatives thereof, and mixtures thereof.

4. The lipophilic topical composition as claimed in claim 1, wherein the pharmaceutically acceptable excipient is selected from the group comprising of carriers, skin penetration enhancers, pharmaceutical surfactants, solubility enhancers, oil phase components, aqueous phase components, emulsifiers, moisturizers, antioxidants, vitamins, lubricants, preservatives, stabilizers, buffers.

5. The lipophilic topical composition as claimed in claim 4, wherein the carriers is selected from the group comprising of dimethyl sulfoxide (DMSO), water, glycerin, petrolatum, stearic acid, glycol stearate, dimethicone, isopropyl isostearate, tapioca starch, cetyl alcohol, glyceryl stearate, magnesium aluminum silicate, carbomer, ethylene brassylate, triethanolamine, disodium EDTA, phenoxyethanol, methyl paraben, propyl paraben, ethanol, bio-polymers (e.g., sodium hyaloronate), liposomes, glycerol, propylene glycol, petrolatum water, and one or more pharmaceutically acceptable penetration enhancer.

6. The lipophilic topical composition as claimed in claim 4, wherein the skin penetration enhancer is selected from the group comprising of lipophilic Propylene Glycols, Dimethyl sulfoxide (DMSO), azones, pyrrolidones, alcohols, alkanols, oleic acids, dimethylformamide (DMF), dimethylacetamide (DMAC), fatty alcohols, fatty acid esters, fatty acids, fatty alcohol ethers, terpenes and biologics.

7. The lipophilic topical composition as claimed in claim 4, wherein the pharmaceutical surfactants or solubility enhancers is selected from the group consisting of lauryl alcohol, polyoxyethylene ether, polyoxyethylene glycerol monostearate, stearic acid ester oxygen poly hydrocarbon, vitamin E succinate, polyethylene glycol ester, sorbitan esters, polyoxyethylene castor oil, polyoxyethylene hydrogenated castor oil, poloxamer, organic esters, poly hill dinitrate 80 or its mixture, DMSO, polyvinylpyrrolidones, yloxy (40) stearate).

8. The lipophilic topical composition as claimed in claim 1, wherein the emulsifier is selected from the group consisting of mono glycerides, di-glycerides, triglycerides, cetyl alcohol, Emulcire 61 WL 1349, Gelot 64, oleyl alcohol, polyoxyethylene oleyl ether, PEG-40 stearate, ceteareth-12, ceteareth-20, ceteareth-30, glyceryl stearate, PEG-100 stearate, methyl myristate, isopropyl myristate, Arlacel 165, glyceryl stearate, PEG-100 stearate, steareth-2 and steareth-20, dimethicone copolyol, Polysorbate 20 (Tween 20), cetyl esters wax, Polysorbate 40 (Tween 40), Polysorbate 60 (Tween 60), Polysorbate 80 (Tween 80), lauramide DEA, cocamide DEA, and cocamide MEA, Phospholipid PTC, alginate, carrageenan, Glucate DO, methylcellulose, polyvinyl alcohol, Carbopol and Carbomer, cetostearyl alcohol, stearic acid, magnesium stearate, sodium lauryl sulfate, triethanolamine, magnesium aluminum silicate, or a combination thereof.

9. The lipophilic topical composition as claimed in claim 4, wherein the pH of the composition is maintained by adding a predetermined quantity of pH modifying agent during the manufacturing step of the composition, wherein the pH adjusting agent is selected from suitable aqueous or non-aqueous pH adjusting agents.

10. The lipophilic topical composition as claimed in claim 1, wherein the ratio of apremilast to lipophilic agent in the composition is 1:100 to 100:1, preferably 1:5 to 10:50.

11. The lipophilic topical composition as claimed in claim 1, wherein the concentration of apremilast in the composition is in the range of 1-10% w/w and the concentration of lipophilic agent is in the range of 2-50% w/w.

12. A lipophilic topical composition of Apremilast, wherein the composition is comprising of Apremilast and at least one lipophilic agent, wherein the composition is prepared by process comprising of:
i. dissolving apremilast in a suitable carrier;
ii. heating at least one lipophilic agent or a mixture of lipophilic agent to 70-75° C.;
iii. adding a preservative or a mixture of preservatives to step ii) lipophilic mixture (oil phase) under stirring;
iv. after solubilization of preservatives, suitable emulsifiers were added under stirring to the step 3 mixture, the temperature of the oil phase was maintained at 70-75° C.;
v. After solubilization of emulsifiers, the temperature of the mixture was decreased to 65-70° C.;
vi. Step i) drug solution was then added to Step v) oil phase under stirring and temperature of the oil phase was maintained at 65-70° C.;
vii. Thereafter suitable carrier was heated to 65-70° C. and added drop wise to the step vi) oil phase under stirring. The stirring was continued till formation of a dispersion;
viii. The pH of the dispersion was adjusted to a pH of about 3-8 using buffering/pH adjusting agent; and
wherein the drug adding step vi) can be performed before or after the formation of dispersion.

13. The lipophilic topical composition as claimed in claim 1, wherein the apremilast is present in the composition with a mixture of two or more lipophilic agents.

14. A method for effectively treating psoriatic symptoms and other related skin disorders by administering a lipophilic composition of Apremilast comprising of Apremilast 1-10% w/w, at least one lipophilic agent 2-50%, emulsifier 2-30%, solvent/carrier 2-40% w/w, preservative 0.05-5% w/w, carrier 10-80% w/w and suitable pH adjusting agent or buffering agent.

15. Use of a lipophilic composition of apremilast for treating psoriasis and other related skin disorders.
